(19) United States
(12) Patent Application Publication
(10) Pub. No.: US 2019/0259476 A1
(43) Pub. Date: Aug. 22, 2019

(54) Patient Consent Systems

(71) Applicant: Sebastian Armijos, Cádiz (ES)

(72) Inventor: Sebastian Armijos, Cádiz (ES)

(21) Appl. No.: 15/901,621

(22) Filed: Feb. 21, 2018

(51) Int. Cl. G16H 10/60; G06F 17/30

(52) U.S. Cl. CPC G16H 10/60; G06F 3/04842; G06F 17/30979

(57) ABSTRACT

A method, system, or device for attaining express consent from an individual for a medical procedure. The device may include a memory device to store instructions, a display device to display a graphical user interface, and a processing device coupled to the memory device and the display. The processing device may receive a notification message from a user device, where the notification message indicates an electronic consent is pending an approval by a patient. The processing device may display a medical procedure presentation to the patient using the display device. The processing device may generate the graphical user interface to display to the patient using the display device, where the graphical user interface includes a consent object associated with a medical procedure. The processing device may receive an input from the patient at the consent object.

### BACKGROUND

When medical procedures are performed, a hospital or medical office may require a patient to consent to the procedure and any risks associated with the procedure prior to the medical procedure. The consent may be express consent or implied consent. A patient may give implicit consent to a medical procedure by his or her situation and/or actions. For example, when the patient is unconscious or it is a medical emergency, the consent may be implied by the situation. While implicit consent may be deemed acceptable in certain situations and/or actions, the medical community requires express consent when possible to avoid any misunderstandings and avoid legal liabilities associated with the medical procedure. To ensure express consent is given by a patient of a medical procedure, a hospital or medical office may require a patient to read and sign an informed consent form indicating their agreement to the medical procedure and acceptance of the associated risks.

### SUMMARY

A method, system, or device for attaining express consent from an individual for a medical procedure. The device may include a memory device to store instructions, a display device to display a graphical user interface, and a processing device coupled to the memory device and the display. The processing device may receive a notification message from a user device, where the notification message indicates an electronic consent is pending an approval by a patient. The processing device may display a medical procedure presentation to the patient using the display device, where the medical procedure presentation includes information associated with a medical procedure to be performed on the patient. The processing device may generate the graphical user interface to display to the patient using the display device, where the graphical user interface includes a consent object associated with the medical procedure. The processing device may receive an input from the patient at the consent object.

### BRIEF DESCRIPTION OF THE DRAWINGS

The present description will be understood more fully from the detailed description given below and from the accompanying drawings of various embodiments of the present embodiment, which is not to be taken to limit the present embodiment to the specific embodiments but are for explanation and understanding.

FIG. 1A illustrates a flowchart for a method to acquire express consent from an individual for a medical procedure, according to an embodiment.

FIG. 1B illustrates a continuation of the flowchart in FIG. 1A for the method to acquire express consent from the individual for the medical procedure, according to an embodiment.

FIG. 2 is a block diagram of a user device in which embodiments of the user device may be implemented in patient consent systems, according to an embodiment.

### DETAILED DESCRIPTION

The disclosed patient consent systems will become better understood through a review of the following detailed description in conjunction with the figures. The detailed description and figures provide merely examples of the various embodiments described herein. Those skilled in the art will understand that the disclosed examples may be varied, modified, and altered and not depart from the scope of the embodiments described herein. Many variations are contemplated for different applications and design considerations; however, for the sake of brevity, the contemplated variations may not be individually described in the following detailed description.

Throughout the following detailed description, examples of various patient consent systems are provided. Related features in the examples may be identical, similar, or dissimilar in different examples. For the sake of brevity, related features will not be redundantly explained in multiple examples. Instead, the use of related feature names will cue the reader that the feature with a related feature name may be similar to the related feature in an example explained previously. Features specific to a given example will be described in that particular example. The reader is to understand that a given feature need not be the same or similar to the specific portrayal of a related feature in any given figure or example.

Prior to medical personnel performing a medical procedure, a hospital or medical office may require a patient to expressly consent to the medical procedure and any risks associated with the medical procedure. The express consent may be required prior to the medical personnel performing the medical procedure to avoid any misunderstandings about what medical procedure is to be performed and avoid legal liabilities associated with a medical procedure.

Conventionally, three elements may be required to establish legally-sufficient informed and express consent. First, the information being consented to is presented in sufficiently understandable terms for the patient to understand what is being consented to. Second, the patient is competent to give consent. Third, the consent is not coerced.

These legal requirements may be burdensome for an individual at a hospital or medical office to meet. Complying with the legal requirements of consent may be time-consuming, where a medical personnel may spend more time obtaining express consent than performing the medical operation that the consent is for. Additionally, the legal requirements may vary from state to state and/or may change over time, further increasing the amount of time to obtain the express consent and making it increasingly difficult to meet the necessary legal requirements to ensure the express consent is legally acceptable in a court of law.

For example, conventionally, to ensure express consent is given by the patient for the medical procedure, the hospital or the medical office may require the patient to read and sign an informed consent form indicating their agreement to the medical procedure and acceptance of the associated risks. To remain legally compliant, an administrator may regularly monitor for changes in the legal requirements for expressed consent. When a change in the legal requirements occurs, the administrator may update the form(s) patients fill out to give express consent each time a change occurs and retrain medical personnel on how to obtain the express consent.

The embodiments described herein may address the above-noted deficiencies by providing a patient consent system to obtain express consent from an individual. The patient consent system may include a first user device to display a graphical user interface for medical personnel to select a medical procedure presentation, a second user device for a patient to view the medical procedure presentation and provide consent, and a storage device to store an electronic version of the consent. The medical procedure presentation may include one or more multimedia objects to improve a comprehension level by the patient and decrease an anxiety level of the patient. The medical procedure presentation may also be standardized for a given medical procedure to conform to current legal requirements for express consent and avoid variations in the information presented to a patient by medical personnel. The medical procedure presentation and the electronic consent obtained from the patient may be stored at a storage device for review by the medical personnel prior to the medical procedure and/or for subsequent access in a legal proceeding.

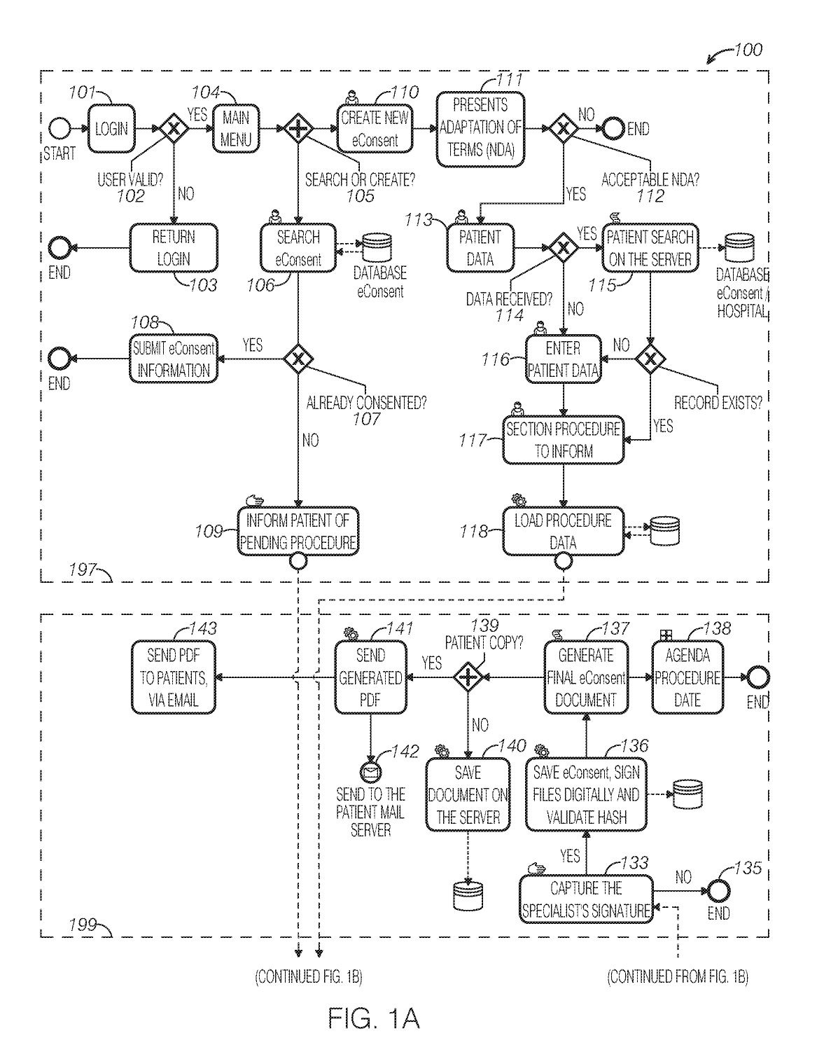

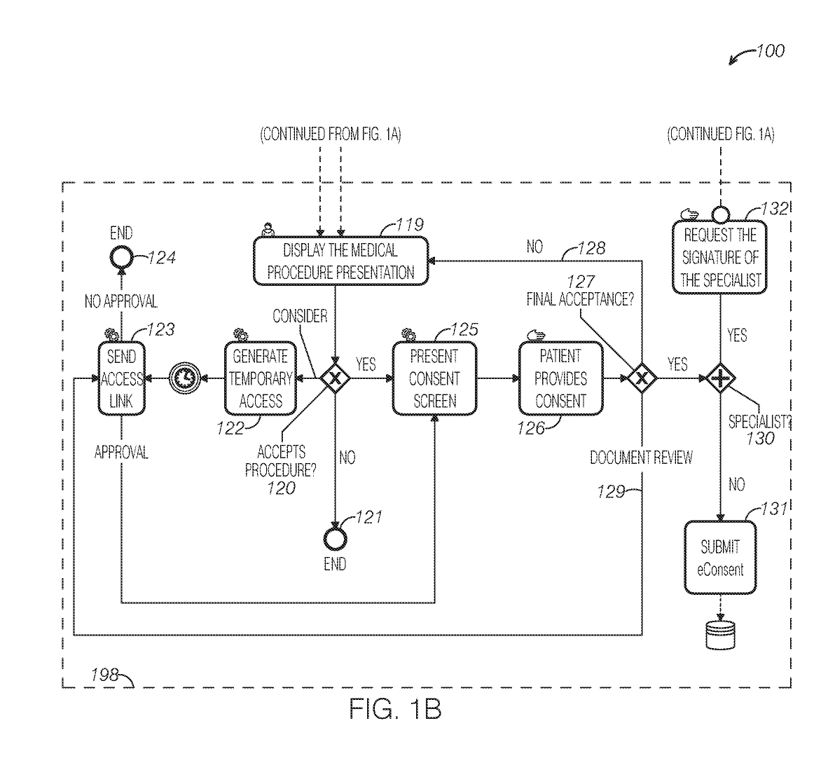

FIG. 1A illustrates a flowchart 100 for a method to acquire express consent from an individual for a medical procedure, according to an embodiment. The method may be performed, at least in part, by a processing device. The processing device may include one or more processors, central processing units (CPUs), integrated circuits, control units, arithmetic and logic units (ALUs), and so forth. The method may include a medical personnel portion 197, a patient portion 198 (as shown in FIG. 1B), and a medical specialist portion 199. The medical personnel portion 197 of the method may begin with a medical personnel logging into a software application (block 101). The medical personnel may be a doctor, a nurse, a medical administrator, a medical staff member, and so forth. In one embodiment, the software application may be executed on a processing device. In another embodiment, the software application may be executed on a virtual machine, a cloud system, or a remote server. In another embodiment, the software application may be a mobile device application, a computer application, an online website, an intranet application, an executable file executing on the processing device, and so forth that is coupled to the processing device.

When the medical personnel logs into the software application, the software application may display a login screen with a graphical user interface (GUI) that includes an identification (ID) input object to receive II) information. The input object may be a button, a text input field, a number input field, radio selection buttons, a submenu, and so forth. In one embodiment, the identification information may include a name of a patient, a number associated with the patient, a name or number associated with a medical procedure, a name of the medical personnel, a number associated with the medical personnel, and so forth. The processing device executing the software application may receive the identification information from an input device. The input device may be a touch screen, a keyboard, a mouse, a microphone, a camera, and so forth that is coupled to the processing device.

The software application may determine whether the identification information is valid (block 102). To determine whether the identification information is valid, the software application may compare the identification information from the ID input object with identification information stored at a storage device. The storage device may be a non-transitory computer-readable medium. The non-transitory computer-readable medium may be a floppy disk, an optical disk, a CD-ROM, a magnetic-optical disk, a read-only memory (ROM), a random access memory (RAM), an EPROM, an EEPROM, a magnetic or optical card, or any type of media suitable for storing electronic instruction or data. When the identification information from the II) input object does not match the identification information stored at a storage device, the identification information may be invalid and the software application may return to the login screen of the software application (block 103).

When the identification information from the ID input object matches the identification information stored at a storage device, the software application may display main menu screen with a GUI (block 104). The GUI may include a first input object for the medical personnel to select a search function and a second input object for a user to select an electronic consent generation function (block 105). When the medical personnel selects the first input object and provide search information, the software application may receive the search information and search a database for a data set corresponding to an electronic consent for a medical procedure and risks corresponding to a medical procedure presentation (106). The medical procedure presentation may include multimedia objects, such as text, audio, video, interactive objects in a graphical user interface, and so forth. For example, the medical procedure presentation may include one or more videos and/or audio segments illustrating one or more portion of the medical procedure. In another example, the medical procedure may include text that describes one or more risks associated with the medical procedure.

When the software application does not find the data set, the software application may return to the main menu screen. When the software application identifies the data set that matches the search information, the software application may determine whether the patient has previously provided consent for the medical procedure presentation (block 107).

When the patient has previously provided consent for the medical procedure and the risks, the software application may send the consent information to the medical personnel (block 108). When the patient has not previously provided consent for the medical procedure and the risks, the software application may send a notification message to a patient's user device (block 109). The notification message may include a message notifying the patient that a medical procedure presentation is awaiting the patient's review and requesting consent to the medical procedure and risks corresponding to the medical procedure presentation. In one embodiment, the notification message may include the medical procedure presentation. In another embodiment, the notification message may include a link or Internet protocol (IP) address for the patient to access the medical procedure presentation and provide consent.

When the medical personnel selects the second input object, the software application may generate a medical procedure presentation (block 110). To generate the medical procedure presentation, the software application may generate a non-disclosure agreement (NDA) (block 111). When the software application generates the NDA, the medical personnel or another individual, such as an attorney, may review the NDA for accuracy and completeness (block 112). The NDA may be selected from a library of NDAs stored in a database or generated from information provided by the medical personnel. In one embodiment, when the medical personnel or the other individual determines that the NDA is not accuracy or is not complete, the software application may terminate. In another embodiment, when the medical personnel or the other individual determines that the NDA is not accuracy or is not complete, the software application may return to the main menu screen

When the medical personnel or the other individual determines that the NDA is accuracy and complete, the software application may continue to generate the medical procedure presentation by requesting the medical personnel input patient data via an input device of the user device (block 113). The software application may determine whether the processing device receives patient data from the input device (block 114), When the processing device receives the patient data, the software application may use the patient data to determine whether the patient exists in a medical database (block 115). When a record of the patient does not exist in the medical database or the processing device does not receive the patient data from the input device, the software application may request the medical personnel input the patient data via one or more input objects in the a GUI (block 116). When the patient does exist in the medical database or the medical personnel inputs the patient data, the software application may request the medical personnel input the medical procedure identification information via one or more input objects in the GUI (block 117). The medical procedure identification information may indicate the type of medical procedure the patient is to undergo.

The software application may use the patient data and the procedure identification information to identify a medical procedure presentation associated with the patient data and the procedure identification information and send the medical procedure presentation to a patient's user device (block 118). In one embodiment, the medical procedure presentation may be standardized presentation for a given medical procedure to conform to current legal requirements for express consent and avoid variations in the information presented to a patient by medical personnel.

In one embodiment, the patient's user device may be the same device as the medical personnel's user device. For example, the user device may be a laptop, tablet device, or smartphone that the medical personnel uses to perform the medical personnel portion 197 of the flowchart 100 and then the medical personnel may give the user device to the patient to perform the patient portion 198 of the flowchart 100, as discussed below. In another embodiment, the patient's user device may be a different device than the user device used by the medical personnel. For example, the user device may be a laptop, a tablet device, or a smartphone that the medical personnel uses to perform the medical personnel portion 197 of the flowchart 100. When the medical personnel completes the medical personnel portion 197, the medical personnel's user device may send the medical procedure presentation to the patient's user device for the patient to complete the patient portion 197 of the flowchart 100, as discussed below.

FIG. 1B illustrates a continuation of the flowchart 100 in FIG. 1A for the method to acquire express consent from the individual for the medical procedure, according to an embodiment. When the patient's user device receives the medical procedure presentation from the medical personnel's user device, the patient's user device may execute a software application to display the medical procedure presentation to the patient as part of a medical procedure presentation screen (block 119). In one embodiment, the software application may verify a user's interaction level or attention level with the medical procedure presentation. For example, the patient's user device may include a sensor, such as a still camera or a video camera, to monitor the user's interaction level or attention level with the medical procedure presentation. In this example, the software application may use sensor data captured by the sensor to determine an amount of time or percentage of time the user watches videos in the medical procedure presentation, an amount of time or percentage of time the user reads text in the medical procedure presentation, an amount of time or percentage of time the user looks at pictures in the medical procedure presentation, and so forth.

In another example, the software application may display a GUI with an input object, such as a button at one or more times during the medical procedure presentation that requests the user to press the button to indicate that the user is paying attention to the medical procedure presentation. In another example, to determine the user's interaction level or attention level, the software application may monitor for at least one of a keyboard entry from an input device, a cursor input from the input device, a mouse click from the input device, or an eye movement input from the input device.

When the user's interaction level or attention level with the medical procedure presentation is below a threshold level, the software application may require the patient to review the medical procedure presentation again to ensure the patient understands the medical procedure and the risks associated with the medical procedure.

When the software application has completed displaying the medical procedure presentation to the patient, the software application may display an input object for the patient to indicate whether he or she agrees to the medical procedure to be performed and accepts the risks associated with the medical procedure (block 120). In one embodiment, the patient may provide consent for himself or herself. In another embodiment, when the patient is not legally able to provide consent (such as a handicapped individual, a minor, or an incapacitated individual) the software application may receive consent from an authorized individual. For example, the software application may receive the consent from a parent, guardian, or an individual with a power of attorney for the individual. To determine whether the patient is able to provide consent, the software program may provide an input object where the individual may indicate whether they are capable of providing consent. For example, the input object may request the patient input they date of birth or mental capacity and the software application may determine whether the patient is the legal age and/or legal mental capacity to give consent. When the patient indicates, via the input object, that he or she does not agree to the medical procedure being performed and/or does not accept the risks associated with the medical procedure, the software application may send a notification message to the medical personnel indicating that the patient does not consent and then the software application may terminate (block 121).

When the patient indicates, via the input object, that he or she would like to consider whether to agree to the medical procedure being performed and/or consider whether to accept the risks associated with the medical procedure, the software application may set a threshold period of time for the patient to consider whether they would like to consent or not consent (block 122). In one example, the threshold period of time may be a maximum period of 48 hours. In another example, the threshold period of time may be a standard amount of time established by medical regulations or legal regulations associated with medical consent.

The software application may save a progress level of the patient's review of the medical procedure presentation and send an access link or access information to the patient's user device for the patient to use to access the software application and indicate whether he or she consents or does not consent (block 123). When the threshold amount of time expires without the patient accessing the software application and indicating whether they are consenting or not consenting, the software application may terminate (block 124). When the software application terminates, the medical personnel may have to resend the medical procedure presentation to the patient's user device to reinitiate the software application on the patient's user device. In another example, when the threshold period of time expires, the software application may require the patient to review the medical procedure presentation again prior to consenting or not consenting.

When the patient provides consent within the threshold amount or provides consent after viewing the medical procedure presentation, the software application may present a graphical user interface with an input object to receive consent information (block 125). In one embodiment, the consent information may include a signature, such as a digital signature or a handwritten signature captured by a touchscreen device. In another embodiment, the consent information may include an audio recording or a video recording capturing a vocal consent by the patient via a microphone and/or camera of the user device. In one example, the consent information may include a signature from the patient and a voice recording from the patient consenting to the medical procedure shown in a video and a risk indicated in the text of the medical procedure presentation. In another embodiment, the consent information may include biometric information. The biometric information may include a fingerprint, a voice print, an eye print or iris print, face print, earlobe print, and so forth.

In one embodiment, the consent information may be representative of a single consent from the patient for the medical procedure and the risks as a whole. In another embodiment, the consent information may be representative of multiple consents from the patient for different parts of the medical procedure and/or different risks associated with the medical procedure. In one example, the consent information may include a first signature from the patient and a first voice recording from the patient consenting to the medical procedure shown in a video of the medical procedure presentation and a second signature from the user and a second voice recording from the user consenting to the risk indicated in the text of the medical procedure presentation. In another example, the medical procedure presentation may include a first video showing a first portion of the medical procedure and a second vide© showing a second portion of the medical procedure.

The consent information may include a first signature from the user and a first voice recording from the user consenting to the first portion medical procedure shown in the first video and a second signature from the user and a second voice recording from the user consenting to the second portion of the medical procedure shown in the second video. In another example, the consent information may include at least one consent to the medical procedure or the risk of the medical procedure and at least one denial of consent to the medical procedure or the risk of the medical procedure. As discussed above, when the patient does not meet the legal requirements to provide consent, a parent, a guardian, or other authorized individual may provide the consent information on behalf of the patient for the medical procedure.

The software application may determine when the input object of the graphical user interface receives the consent information (block 126). When the software application receives the consent information, the software application may display another input object via the graphical user interface to confirm final consent of the medical procedure and the associated risks (block 127).

When the patient does not give final consent, the software application may return to the medical procedure presentation display screen (arrow 128). When the patient desires more time to review the medical procedure presentation, the software application may send the access link or the access information for the patient to have the threshold amount to further consider the medical procedure and the associated risks (arrow 129). When the patient provides final consent via the input object, the software application may determine whether a medical specialist is needed (block 130). In one example, the medical procedure presentation may indicate whether a medical specialist is required for the medical procedure. In another example, the software application may display a specialist page with an input object where the patient may request a. second opinion from a specialist.

When the medical procedure presentation indicates that a medical specialist is not required or the patient does not request a second opinion, the software application may store the consent information from the patient in a database (block 131). The software application may also send a notification message to the medical personnel's user device to inform the medical personnel that the patient has consented to the medical procedure and the associated risks. When the medical procedure presentation indicates that a medical specialist is required or the patient requests a second opinion, the software application may generate a consent form and send the consent form to the medical specialist's user device (block 132).

In one embodiment, the specialist's user device may be the same device as the medical personnel and/or the patient's user device. For example, the user device may be a laptop, tablet device, or smartphone that the patient uses to perform the patient portion 198 of the flowchart 100 and then the patient may give the user device to the medical specialist to perform the medical specialist portion 199 of the flowchart 100, as discussed below. In another embodiment, the medical specialist's user device may be a different device as the user device used by the medical personnel. For example, the user device may be a laptop, tablet device, or smartphone that the medical personnel uses to perform the medical personnel portion 197 of the flowchart 100. When the medical personnel completes the medical personnel portion 197, the medical personnel's user device may send the medical procedure presentation to the patient's user device for the patient to complete the patient portion 198 of the flowchart 100, as discussed below.

Returning to the medical specialist portion 199 of FIG. 1A, a software application executing on the medical specialist's user device may receive the consent form and display a specialist consent screen with the consent form that includes a consent object for the medical specialist to provide his or her consent to the medical procedure and the associated risks (block 133). When the medical specialist does not provide their consent, the software application may terminate (block 135). In one example, when the software application terminates, the software application may send a notification message to the medical personnel and/or patient's user device(s) indicating that the medical specialist did not provide his or her consent.

When the medical specialist does not provide their consent, the software application may save the consent information to a database (block 136). In one example, the consent information may include a digital signature, a signature captured via a touch screen, hash identification information (such as a passcode or pin code), a multimedia recording, and so forth. In one embodiment, the software application may validate the medical specialist's consent information to verify the consent is from the medical specialist. When the consent information has been received, the software application may generate a final consent document that includes one or more of the medical procedure presentation, the patient's consent information, and/or the medical specialist's consent information (block 137).

In one embodiment, when the final consent document has been generated, the software application may schedule the medical procedure for a procedure date (block 138). In one example, the procedure date may be predefined by the medical specialist, the patient, and/or the medical personnel and the software application may send out a meeting invitation notification message to the medical specialist's, the patient's, and/or the medical personnel's user devices for the medical specialist, the patient, and/or the medical personnel to accept. In another embodiment, the software application may automatically schedule the procedure date according to an electronic schedule of the medical specialist, the patient, and/or the medical personnel.

In another embodiment, the software application may determine whether to send a copy of the final consent document to the patient (block 139). In one embodiment, the software application may send a request to the patient's user device inquiring whether the patient would like to receive the final consent document. In another embodiment, the final consent document may be sent to the patient or not sent to the patient based on a predefined permission of the medical personnel, the patient, and/or the medical specialist. For example, when the medical specialist provides his or her consent, the medical specialist may indicate whether to send the final consent document to the patient or keep the final consent document confidential to the medical specialist and/or the medical personnel.

When the software application determines that a copy of the final consent document is not to be sent to the patient, the software application may store the final consent document at a storage device, such as a server (block 140). When the software application determines that a copy of the final consent document is to be sent to the patient, the software application may generate a final consent document, such as a portable document format (PDF) document (block 141). The software application may determine how to send the final consent document to the patient based on a predefined preference by the patient. In one embodiment, the software application may send the final consent document to a storage device for the patient, such as a cloud-based storage device or server (block 142). In another embodiment, the software application may send the final consent document to an electronic mail (e-mail) account associated with the patient (block 143). When the software application has sent the final consent document to the patient, the software application may terminate.

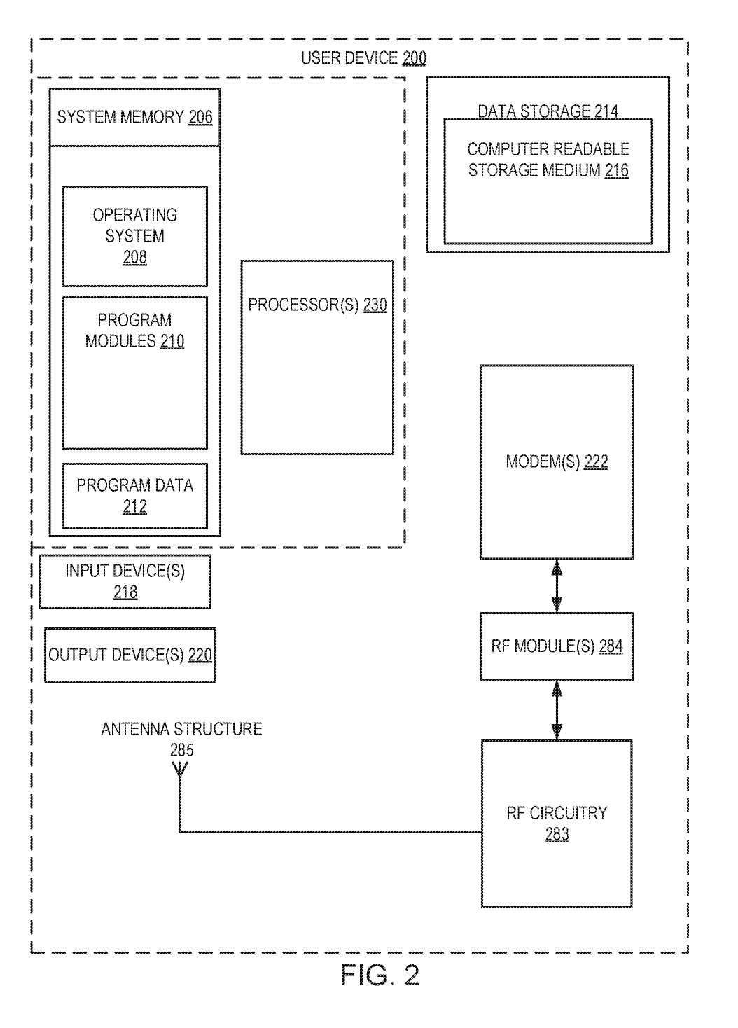

FIG. 2 is a block diagram of a user device 200 in which embodiments of the user device 200 may be implemented in patient consent systems, according to an embodiment. The user device 200 may correspond to the user device discussed in FIGS. 1A and 1B. In one embodiment, the user device 200 may be any type of computing device such as an electronic book reader, a PDA, a mobile phone, a laptop computer, a portable media player, a tablet computer, a camera, a video camera, a netbook, a desktop computer, a gaming console, a DVD player, a computing pad, a media center, and the like. In another embodiment, the user device 200 may be an Internet of things (IoT) device. The IoT device may be computing device that connects wirelessly to a network, such as the Internet, and is configured to transmit or receive data over the network with another device.

The user device 200 may be any portable or stationary user device. For example, the user device 200 may be an intelligent voice control and speaker system. Alternatively, the user device 200 can be any other device used in a WLAN network (e. Wi-Fi® network), a WAN network, or the like.

The user device 200 includes one or more processor(s) 230, such as one or more CPUs, microcontrollers, object programmable gate arrays, or other types of processors. The user device 200 also includes system memory 206, which may correspond to any combination of volatile and/or non-volatile storage mechanisms. The system memory 206 stores information that provides operating system component 208, various program modules 210, program data 212, and/or other components. In one embodiment, the system memory 206 stores instructions methods as described herein. The user device 200 performs functions by using the processor(s) 230 to execute instructions provided by the system memory 206.

The user device 200 also includes a data storage device 214 that may be composed of one or more types of removable storage and/or one or more types of non-removable storage. The data storage device 214 includes a computer-readable storage medium 216 on which is stored one or more sets of instructions embodying any of the methodologies or functions described herein. Instructions for the program modules 210 may reside, completely or at least partially, within the computer-readable storage medium 216, system memory 206 and/or within the processor(s) 230 during execution thereof by the user device 200, the system memory 206 and the processor(s) 230 also constituting computer-readable media. The user device 200 may also include one or more input devices 218 (keyboard, mouse device, specialized selection keys, etc.) and one or more output devices 220 (displays, printers, audio output mechanisms, etc.).

The user device 200 further includes modem 222 to allow the user device 200 to communicate via a wireless network(s) (e.g., such as provided by the wireless communication system) with other computing devices, such as remote computers, an item providing system, and so forth. The modern 222 can be connected to zero or more RF modules 284. The zero or more RF modules 284 can be connected to RF circuitry 283. The RF modules 284 and/or the RF circuitry 283 may be a WLAN module, a WAN module, PAN module, or the like. The modem 222 allows the user device 200 to handle both voice and non-voice communications (such as communications for text messages, multimedia messages, media downloads, web browsing, etc.) with a wireless communication system. The modem 222 may provide network connectivity using any type of mobile network technology including, for example, cellular digital packet data (CDPD), general packet radio service (CPRS), EDGE, universal mobile telecommunications system (UMTS), 1 times radio transmission technology (1×RTT), evaluation data optimized (EVDO), high-speed downlink packet access (HSDPA), Wi-Fi® technology, Long Term Evolution (LTE) and LTE Advanced (sometimes generally referred to as 4G), etc.

The modem 222 may generate signals and send these signals to the antenna structure 285 via the RF modules 284 and the RF circuitry 283 as described herein. User device 200 may additionally include a WLAN module, a GPS receiver, a PAN transceiver and/or other RF modules.

In one embodiment, the user device 200 establishes a first connection using a first wireless communication protocol, and a second connection using a different wireless communication protocol. The first wireless connection and second wireless connection may be active concurrently, for example, if a user device is downloading a media item from a server (e.g., via the first connection) and transferring a file to another user device (e.g., via the second connection) at the same time. Alternatively, the two connections may be active concurrently during a handoff between wireless connections to maintain an active session (e.g., for a telephone conversation). Such a handoff may be performed, for example, between a connection to a WLAN hotspot and a connection to a wireless carrier system. In one embodiment, the first wireless connection is associated with a first resonant mode of an antenna structure that operates at a first frequency band and the second wireless connection is associated with a second resonant mode of the antenna structure that operates at a second frequency band. In another embodiment, the first wireless connection is associated with a first antenna element and the second wireless connection is associated with a second antenna element. In other embodiments, the first wireless connection may be associated with a media purchase application (e.g., for downloading electronic books), while the second wireless connection may be associated with a wireless ad hoc network application. Other applications that may be associated with one of the wireless connections include, for example, a game, a telephony application, an Internet browsing application, a file transfer application, a global positioning system (GPS) application, and so forth.

Though modem 222 is shown to control transmission and reception via the antenna structure 285, the user device 200 may alternatively include multiple modems, each of which is configured to transmit/receive data via a different antenna and/or wireless transmission protocol.

The user device 200 delivers and/or receives items, upgrades, and/or other information via the network. For example, the user device 200 may download or receive items from an item providing system. The item providing system receives various requests, instructions and other data from the user device 200 via the network. The item providing system may include one or more machines (e.g., one or more server computer systems, routers, gateways, etc.) that have processing and storage capabilities to provide the above functionality. Communication between the item providing system and the user device 200 may be enabled via any communication infrastructure. One example of such an infrastructure includes a combination of a wide area network (WAN) and wireless infrastructure, which allows a user to use the user device 200 to purchase items and consume items without being tethered to the item providing system via hardwired links. The wireless infrastructure may be provided by one or multiple wireless communications systems, such as one or more wireless communications systems. One of the wireless communication systems may be a wireless local area network (WLAN) hotspot connected to the network. The WLAN hotspots can be created by products based on IEEE 802.11x standards for the Wi-Fi® technology by Wi-Fi® Alliance. Another of the wireless communication systems may be a wireless carrier system that can be implemented using various data processing equipment, communication towers, etc. Alternatively, or in addition, the wireless carrier system may rely on satellite technology to exchange information with the user device 200.

The communication infrastructure may also include a communication-enabling system that serves as an intermediary in passing information between the item providing system and the wireless communication system. The communication-enabling system may communicate with the wireless communication system (e.g., a wireless carrier) via a dedicated channel, and may communicate with the item providing system via a non-dedicated communication mechanism, e.g., a public Wide Area Network (WAN) such as the Internet.

The user device 200 are variously configured with different functionality to enable consumption of one or more types of media items. The media items may be any type of format of digital content, including, for example, electronic texts (e.g., eBooks, electronic magazines, digital newspapers, etc.), digital audio (e.g., music, audible books, etc.), digital video (e.g., movies, television, short clips, etc.), images (e.g., art, photographs, etc.), and multi-media content. The user device 200 may include any type of content rendering devices such as electronic book readers, portable digital assistants, mobile phones, laptop computers, portable media players, tablet computers, cameras, video cameras, netbooks, notebooks, desktop computers, gaming consoles, DVD players, media centers, and the like.

In the above description, numerous details are set forth. It will be apparent, however, to one of ordinary skill in the art having the benefit of this disclosure, that embodiments may be practiced without these specific details. In some instances, well-known structures and devices are shown in block diagram form, rather than in detail, in order to avoid obscuring the description.

Some portions of the detailed description are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like.

It should he borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “inducing,” “parasitically inducing,” “radiating,” “detecting,” determining,” “generating,” “communicating,” “receiving,” “disabling,” or the like, refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices.

Embodiments also relate to an apparatus for performing the operations herein. This apparatus may be specially constructed for the required purposes, or it may comprise a general-purpose computer selectively activated or reconfigured by a computer program stored in the computer. Such a computer program may be stored in a computer-readable storage medium, such as, but not limited to, any type of disk including floppy disks, optical disks, CD-ROMs and magnetic-optical disks, read-only memories (ROMs), random access memories (RAMS), EPROMs, EEPROMs, magnetic or optical cards, or any type of media suitable for storing electronic instructions.

The algorithms and displays presented herein are not inherently related to any particular computer or other apparatus. Various general-purpose systems may be used with programs in accordance with the teachings herein, or it may prove convenient to construct a more specialized apparatus to perform the required method steps. The required structure for a variety of these systems will appear from the description below. in addition, the present embodiments are not described with reference to any particular programming language. It will be appreciated that a variety of programming languages may be used to implement the teachings of the present invention as described herein. It should also be noted that the terms “when” or the phrase “in response to,” as used herein, should be understood to indicate that there may be intervening time, intervening events, or both before the identified operation is performed.

It is to be understood that the above description is intended to be illustrative, and not restrictive. Many other embodiments will be apparent to those of skill in the art upon reading and understanding the above description. The scope of the present embodiments should, therefore, be determined with reference to the appended claims, along with the full scope of equivalents to which such claims are entitled.

The disclosure above encompasses multiple distinct embodiments with independent utility. While these embodiments have been disclosed in a particular form, the specific embodiments disclosed and illustrated above are not to be considered in a limiting sense as numerous variations are possible. The subject matter of the embodiments includes the novel and non-obvious combinations and sub-combinations of the various elements, features, functions and/or properties disclosed above and inherent to those skilled in the art pertaining to such embodiments. Where the disclosure or subsequently filed claims recite “a” element, “a first” element, or any such equivalent term, the disclosure or claims is to be understood to incorporate one or more such elements, neither requiring nor excluding two or more such elements.

Applicant(s) reserves the right to submit claims directed to combinations and sub-combinations of the disclosed embodiments that are believed to be novel and non-obvious. Embodiments embodied in other combinations and sub-combinations of features, functions, elements and/or properties may be claimed through amendment of those claims or presentation of new claims in the present application or in a related application. Such amended or new claims, whether they are directed to the same embodiment or a different embodiment and whether they are different, broader, narrower or equal in scope to the original claims, are to be considered within the subject matter of the embodiments described herein.

1. A method, comprising:
receiving, by a graphical user interface, identification information from a first individual;
verifying, by a processing device, that the identification information is associated with an identity of a patient or a doctor;
displaying, by the graphical user interface, a first graphical display that includes a first input object and a second input object, wherein the first input object corresponds to a search function to search for a first data set corresponding to an electronic consent for a medical procedure and the second input object corresponds to an electronic consent generation function;
in response to receiving a first input at the first input object, identifying the first data set in a database corresponding to the first input if the database is storing the first data set;
in response to receiving a second input at the second input object, generating a second data set corresponding to the second input, wherein the second data set corresponds to the electronic consent for the medical procedure;
sending, by the processing device, a notification message to a user device, wherein the notification message indicates the electronic consent is pending an approval by a user;
displaying, by the user device, a medical procedure presentation to the user, wherein the medical procedure presentation includes information associated with the medical procedure;
displaying, by the user device, a consent object to the user to consent to the medical procedure;
receiving, by the user device, a third input from the user, wherein the third input indicates whether the user consents to the medical procedure or does not consent to the medical procedure; and
in response the user consenting to the medical procedure, storing the third input in a data storage device.

2. The method of claim 1, wherein the medical procedure presentation comprises:
a video showing at least a portion of the medical procedure; and
text indicating a risk associated with the medical procedure.

3. The method of claim 2, wherein the third input includes a signature from the user and a voice recording from the user consenting to the medical procedure shown in the video and the risk indicated in the text.

4. The method of claim 2, wherein the third input includes:
a first signature from the user and a first voice recording from the user consenting to the medical procedure shown in the video; and
a second signature from the user and a second voice recording from the user consenting to the risk indicated in the text.

5. The method of claim 1, wherein the medical procedure presentation comprises:
a first video showing a first portion of the medical procedure; and
a second video showing a second portion of the medical procedure.

6. The method of claim 5, wherein the third input includes:
a first signature from the user and a first voice recording from the user consenting to the first portion medical procedure shown in the first video; and
a second signature from the user and a second voice recording from the user consenting to the second portion of the medical procedure shown in the second video.

7. The method of claim 1, further comprising verifying the user interacted with the medical procedure presentation.

8. The method of claim 1, further comprising in response to the user consenting to the medical procedure, sending a second notification message to a second user device associated with the doctor, wherein the second notification message indicates the user consenting to the medical procedure.

9. The method of claim 1, wherein the identification information comprises at least one of a name of the patient, a number associated with the patient, a name or number associated with the medical procedure, a name of the doctor, a number associated with the doctor.

10. The method of claim 1, wherein when the third input is indicative of the consent, the third input comprises a plurality of consents to the medical procedure and a risk of the medical procedure.

11. The method of claim 10, wherein the plurality of consents comprises at least one consent to the medical procedure or the risk of the medical procedure and at least one denial of consent to the medical procedure or the risk of the medical procedure.

12. The method of claim 1, further comprising:
determining the medical procedure requires a specialist to perform at least a portion of the medical procedure;
sending a consent request to the specialist to consent to the medical procedure; and
storing the consent from the specialist at the data storage device.

13. The method of claim 1, further comprising:
determining that the database is not storing the first data set corresponding to the first input;
requesting consent information from the from the first individual; and
generating e first data set using the consent information.

14. A device, comprising:
a memory device to store instructions;
a display device to display a graphical user interface; and
a processing device coupled to the memory device and the display, the processing device to:
receive a notification message from a user device, wherein the notification message indicates an electronic consent is pending an approval by a patient;
display, by the display device, a medical procedure presentation to the patient, wherein the medical procedure presentation includes information associated with a medical procedure to be performed on the patient;
generate the graphical user interface to display to the patient using the display device, wherein the graphical user interface includes a consent object associated with the medical procedure; and
receive an input from the patient at the consent object.

15. The device of claim 14, wherein the input from the patient at the consent object comprises at least one of:
a first input entry indicating that the patient consents to the medical procedure;
a second input entry indicating that the patient does not consent to the medical procedure; or
a third input entry indicating that the patient is delaying a decision to consent or not consent to the medical procedure.

16. The device of claim 14, wherein the medical procedure presentation comprises:
a video showing at least a portion of the medical procedure; and
text indicating a risk associated with the medical procedure.

17. The device of claim 16, wherein the input includes a signature from the patient and a voice recording from the patient consenting to the medical procedure shown in a video of the medical procedure presentation and the risk indicating in text of the medical procedure presentation.

18. A non-transitory computer-readable medium storing instructions to cause a processing device to:
in response to receiving a notification message from a user device, display a medical procedure presentation to a patient, wherein:
the medical procedure presentation includes a first multimedia object associated with a medical procedure to be performed on the patient; and
the medical procedure presentation includes a second multimedia object indicating a risk of the medical procedure;
generate a graphical user interface to display to a user, wherein the graphical user interface includes a consent object associated with the medical procedure;
receive an input from the patient at the consent object, wherein the input indicates whether the user consents to the medical procedure; and
send the input to the user device.

19. The non-transitory computer-readable medium of claim 18, further comprising storing instructions to cause the processing device to monitor a progress of the patient viewing the medical procedure presentation.

20. The non-transitory computer-readable medium of claim 19, wherein to monitor the progress of the patient viewing the medical procedure presentation, the processing device is further to monitor for at least one of a keyboard entry from an input device, a cursor input from the input device, a mouse click from the input device, or an eye movement input from the input device.
